(19) United States
(12) Patent Application Publication
(10) Pub. No.: US 2023/0270660 A1
(43) Pub. Date: Aug. 31, 2023

(54) Silybum marianum (L.) Gaertn. oil for reinforcing the barrier function of the skin

(71) Applicant: PIERRE FABRE DERMO-COSMETIQUE, BOULOGNE-BILLANCOURT (FR)

(72) Inventors: Mathieu LETI, Baziege (FR); Carine JACQUES JAMIN, TOURNEFEUILLE (FR)

(21) Appl. No.: 18/042,679

(22) PCT Filed: Aug. 31, 2021
(86) PCT No.: PCT/FR2021/051499

§ 371 Date: Feb. 23, 2023

(30) Foreign Application Priority Data
Aug. 31, 2020 (FR) FR2008818

(51) Int. Cl. A61K 8/92; A61Q 19/00; A61Q 17/00

(52) U.S. Cl. CPC A61K 8/922; A61Q 17/00; A61Q 19/007

(57) ABSTRACT

The invention relates to the cosmetic use of an oil derived from akenes of Silybum marianum (L.) Gaertn. to prevent a decrease in and/or increase the epidermal barrier function.

### TECHNICAL FIELD OF THE INVENTION

The present invention relates to the use of an oil derived from akenes of Silybum marianum (L.) Gaertn. and/or the use of cosmetic compositions comprising such an oil, as well as a cosmetic method for preventing the decrease in and/or increasing the epidermal barrier function.

### STATE OF THE ART

The scientific name Silybum marianum (L.) Gaertn. designates a plant belonging to the Asteraceae family, which is annual or biennial with a robust stem that can reach more than one meter in height. Its large, shiny, alternating leaves, without stipules, are marbled with white and edged with hard, pointed spines. The flowers are grouped in terminal heads, often solitary. They are surrounded by large spiny bracts with very sharp ends. The flowers, which are tubular, with five lobes, are purplish crimson in color. The fruits are shiny akenes, which are black or marbled with yellow, surmounted by a pappus with bristles which are denticulated in a ring at their base. The main vernacular name for this plant is Milk Thistle. This plant particularly likes dry and sunny places, often on acid, dry and stony soils. Its geographical distribution is concentrated around the Mediterranean, but it is also present in Europe, West Asia, as well as in North America and Australia or even New Zealand. It grows in gardens but is most dominant in uncultivated fields, pastures, along path edges and among rubble.

The akene (often erroneously referred to as seed in the literature) of Silybum marianum (L.) Gaertn. and its preparations are conventionally used orally, in the symptomatic treatment of functional digestive disorders attributed to a hepatic origin.

The main active ingredient of Silybum akene marianum (L.) Gaertn. is silymarin, which is a mixture of several flavonolignans. Silymarin contains mainly (at least 95% by weight) a mixture of the following four flavonolignans: silybin, isosilybin, silychristin and silydianin (Kuki and al., Chromatographia 2012, 75, 175-180). The akenes contain up to 3% by weight of silymarin. They also consist of oil (15-30% by weight), mucilages and proteins.

Silymarin has been the subject of numerous (in vitro, in vivo and clinical) studies having demonstrated its antioxidant, hepatoprotective, digestive and even anti-inflammatory properties. Currently, extracts of akenes of Silybum marianum (L.) Gaertn. titrated in silymarin are present in several pharmaceutical preparations intended for the treatment of various hepatic and biliary disorders, such as Legalon ®.

The antiproliferative effect of silybin was studied in a model of HepG2 cells derived from hepatocarcinoma. Silybin has been shown to induce a significant increase in the synthesis of certain ceramides which can act as a second messenger in different apoptotic processes (Zappavigna and al., Int. J. Mol. Sciences 2019, 20, 2190). An increase in ceramide synthesis was also demonstrated in the same cell model by silybins A and B and synthetic derivatives, 3-O-galloyl silybin A and 3-O-galloyl silybin B (Boojar and al., Iranian J. Pharmaceutical Res. 2016, 15(3), 421-433).

The akenes of Silybum marianum (L.) Gaertn. usually contain 15-30% oil. The elimination of the oil from the akenes (de-oiling) is a preliminary step to the extraction of the silymarin. Silybum marianum (L.) Gaertn. oil is as such a co-product of the production of silymarin (Zhu and al., Biochemidice and Pharmacotherapy 2018, 100, 191-197). Silybum marianum (L.) Gaertn. oil is therefore devoid of silymarin or contains non-detectable traces thereof. This is confirmed by the analysis of the polyphenol fraction of milk thistle oil which indeed does not reveal the presence of any constituent of silymarin (Meddeb and al., Antioxidants, 2018, 7, 95; Zarrouk and al., Current Pharmaceutical Design, 2019, 25, 1791-1805).

Unrefined Silybum marianum (L.) Gaertn. oil is essentially composed of triglycerides of unsaturated fatty acids, the majority of which are linoleic acid (30 to 60%) and oleic acid (15 to 30%). Its high content of unsaturated fatty acids allows it to enter into anti-cholesterol diets and to be used in the prevention of cardiovascular diseases (El - Mallah and al., Grasas y Aceites 2003, 54(4), 397 -402). The oil also contains saturated fatty acids: palmitic acid (5-15%), stearic acid (3-8%), arachidic acid (1-4%) and behenic acid (1-4%). The crude oil obtained by cold pressing also contains phytosterols (beta-sitosterol in particular) and tocopherols (α-tocopherol, and γ-tocopherol in particular) (Dabbour and al., Pakistan Journal of Nutrition 2014, 13(2), 67-78).

Silybum marianum (L.) Gaertn. oil is mainly used in the culinary field.

Moreover, studies on the antioxidant and hepatoprotective properties of Silybum marianum (L.) Gaertn. oil, administered orally, were performed in vivo in rats or mice (Hermenean and al., Open Life Sci. 2015, 10-225-236; Zhu and al., Pharmacogn Mag 2014, 10(Sup 1), S92-S99). Several in tubo or in vitro studies highlight the antioxidant and cytoprotective properties of Silybum marianum (L.) Gaertn. oil obtained by cold pressing or solvent extraction (Dabbour and al., 2014; Harrabi and al., Lipids in Health and Disease 2018, 17, 82; Meddeb and al., Antioxidants 2018 7, 95).

A clinical study highlights the anti-aging effect on aged skin and by repeated topical application of 2 cosmetic formulations containing 1% Silybum marianum (L.) Gaertn. oil. An improvement in facial wrinkles, dermis density, elasticity and skin tone is observed after applications twice a day for 2 weeks. However, these formulations contain several active ingredients such as palmitoyl peptides, vitamin E, jojoba oil, avocado oil, glycosphingolipids, and sodium hyaluronate (Hahn and al., Experimental and Therapeutic Medicine 2016, 12, 1171-1176). The use of Silybum marianum (L.) Gaertn. oil as a biofuel is also considered (Takase and al., Ultrasonics Sonochemistry 2014, 21, 1752-1762).

The skin is made up of different tissues forming a vital barrier for the body vis-à-vis the external environment. This barrier protects the body against external aggressions, in particular chemical, mechanical or infectious, and as such a certain number of defense reactions against environmental factors and/or xenobiotics occur at its level.

The skin is made up of three main parts, a superficial one, the epidermis, the inner part, the dermis and a deeper layer, the hypodermis, which interact.

The human epidermis is made up of four to five distinct layers (depending on the anatomical site) and four types of cells, which are keratinocytes, which are very predominant, melanocytes, Langerhans cells and Merkel cells. Each of these cell types contributes by its own functions to the essential role played in the organism by the skin, in particular the role of protecting the organism from external aggressions. This property is called barrier function.

The epidermal cells proliferate at its deepest layer, the basal layer, and differentiate during their migration towards the upper layers to successively form the spiny layer consisting of several layers of polyhedral cells disposed on the germinal layers, the granular layer made up of flattened cells containing distinct cytoplasmic inclusions, the grains of keratohyaline and finally the horny layer (or stratum corneum) which is the most superficial layer of the epidermis. The horny layer is made up of 20 to 30 layers of keratinocytes at the terminal stage of their differentiation called corneocytes. The corneocytes, constituent elements of the horny layer, are dead, flat cells containing water and keratin. The architecture of the stratum corneum is conventionally likened to a brick wall. The bricks represent the corneocytes. The corneocytes are surrounded by a lipophilic “cement” made up of lipids. The barrier function is mainly ensured by the stratum corneum in its structure and composition. During the differentiation of keratinocytes, the phospholipids, whose role is to develop the fluid structure of the cell membranes of the living layers of the epidermis, are gradually replaced by a mixture composed mainly of fatty acids, cholesterol and sphingolipids (ceramides). These lipids, which are organized into lamellar bilayers, form the intracellular cement of the stratum corneum. The supramolecular organization of intercorneocyte lipids plays a key role in establishing the physicochemical properties of the stratum corneum and consequently in maintaining a physiological water gradient. The structure of these lipid bilayers has particular assembly properties, either hexagonal (gelled state) or orthorhombic (crystalline system whose unit cell is a rectangular parallelepiped), the latter being the majority (Bouwstra and al., Int. J. Cosmet.Sci., 2008, 30, 388). The orthorhombic state represents the densest conformation and a balance between these two states is necessary for optimal barrier properties. A disruption in the proportion of the three families of stratum corneum lipids leads to a modification of the orthorhombic and hexagonal states and consequently a modification of the barrier function. This structure in lamellar bilayers alternates hydrophilic and lipophilic zones which condition the barrier function, the exchanges of water between the organism and the external environment, as well as the hydration of the stratum corneum.

The latter has long been considered a simple layer of dead cells with no real function. In fact, it is metabolically active and largely provides the barrier function of the epidermis.

The epidermis is not irrigated by any blood vessel and is only supplied by diffusion from the dermis.

The dermis provides the epidermis with a solid support. The dermis is a connective tissue composed of different cell types including fibroblasts, lymphocytes and macrophages. Associated with these cells are collagen fibers and elastin, included in a gel called “ground substance”. Collagen and elastin are synthesized by fibroblasts. There are also leukocytes, mast cells or else tissue macrophages. Finally, the dermis is crossed by blood vessels and nerve fibers, in particular sensory fibers free or connected to sensors.

The cohesion between the epidermis and the dermis is ensured by the dermo-epidermal junction. The balance of the skin barrier and mucous membranes is dependent on complex biological mechanisms involving numerous growth factors, hormones, enzymes and mediators within the epidermis and dermis.

Finally, the hypodermis is the deepest and thickest layer of the skin. It is part of the continuity of the dermis without any real separation between the two tissues. The hypodermis forms a cushion that acts as a mechanical protection for the underlying structures. This fatty layer also allows to insulate the body from thermal variations. If the dermis can be considered as a water reserve, the fats stored within the adipocytes of the hypodermis constitute an energy reserve. It is clear that the quality of the skin barrier and mucous membranes is dependent on complex endogenous biological mechanisms involving numerous growth and differentiation factors, adhesion molecules, hormones and lipid metabolism enzymes.

Thus, an alteration of the cutaneous barrier and/or a break in the continuity of the surface of the skin can occur in the presence of external aggressions such as irritating agents (detergents, acids, bases, oxidants, reducers, concentrated solvents, gases or toxic fumes), mechanical stress (friction, shock, abrasion, tearing of the surface, projection of dust, particles, shaving or hair removal), thermal or climatic imbalances (cold, dryness, radiation), or xenobiotics (undesirable microorganisms, allergens) or internal aggressions such as psychological stress. These aggressions cause lipid deficiencies, in particular for ceramides. These changes in the lipid ratio will modify the organization of the lipid cement and lead to an alteration of the barrier function, increasing transepidermal water loss and a modification of the natural hydration factors. These changes will lead to dehydration of the skin and dry skin and may also aggravate cases of atopic dermatitis, sensitive or reactive skin sensations.

This alteration of the cutaneous barrier can in particular result in cutaneous discomfort, sensory phenomena and in particular unpleasant phenomena. This feeling of cutaneous discomfort can be manifested in particular by tingling, tightness, warming, itching. These sensations of skin discomfort are more frequent in the most exposed areas of the body, namely the hands, feet, face and scalp. They can occur in particular on areas subject to certain daily or frequently renewed hygiene gestures such as shaving, hair removal, cleansing with toiletries or household products, the application of adhesives with bandages or patches, the fixing of prostheses or in the case of sporting, professional gestures or simply related to the way of life and the use of clothing, tools or equipment generating localized friction. They can also be amplified by psychological stress.

The alteration of the cutaneous barrier can also promote the appearance of micro-chapping or microcracks, in particular at the hands, feet and lips.

These sensations of cutaneous discomfort concern all people and in particular those who have sensitive or even intolerant skin. The concept of sensitive skin reflects the level of sensitivity of each person’s skin. While it is possible to have sensitive skin at any age, it is extremely common in babies and the elderly. Babies’ skin is about one-fifth the thickness of adult skin. It is therefore extremely sensitive to chemical, physical and microbial aggressions, as well as to UV rays. The barrier function of adult skin, on the other hand, gradually weakens with age, along with the slowing down of metabolic processes. The aging of the skin gradually leads to a deficiency in lipids, which makes it more easily irritated by alkaline substances such as soap.

When the skin has a very low sensitivity threshold, that is to say it reacts excessively to the slightest external aggression, it will be called intolerant skin, or even reactive intolerant skin. Intolerant skins are more vulnerable to external aggressions and are characterized by daily discomfort and strong irritability. Certain signs, more or less marked, allow to recognize them. The intolerant skin of the face has for example redness and tingling, it tightens, warms up or itches, it can also cause burning sensations. Intolerant skins generally have an allergic background and are therefore particularly sensitive to the components of cosmetic care.

Sensitive skin is in fact skin prone to tingling, warming, formication and itching, sometimes accompanied by redness. These feelings of discomfort appear excessively in reaction to stimuli that would not trigger irritation on a skin called normal skin. This hyper-sensitivity of the skin results from a reduction in its tolerance threshold. The more sensitive the skin, the lower its tolerance threshold and when the tolerance threshold is at its lowest, it will be called intolerant skin. This hyper-sensitivity can be explained by different factors, but the most important is an alteration of the barrier function of the epidermis. This phenomenon then promotes dehydration of the skin and especially the penetration of potentially irritating agents.

The skin is covered with a protective film, called hydrolipidic film. It is the outermost barrier, as well as the most fragile, and the most easily disturbed. It consists largely of fatty substances excreted by the sebaceous glands and lipids resulting from the degradation of cells (squalene, waxes, triglycerides, free fatty acids, cholesterol esters) during the keratinization of horny cells, as well as hydrophilic compounds, such as water from sweat, glycerol, urea, natural skin moisturizing factors, salts, metabolites of the skin flora. This surface film is very exposed and very sensitive to environmental stresses, hygiene habits, and the condition of the skin as well as exposure to UV radiation. The microbiota can significantly modify the composition of sebum, degrade triglycerides and modify the ratios of free fatty acids, in particular during stress or illness. It is therefore important to preserve and even improve this skin barrier function, especially for the most sensitive skins.

There is still a need for agents to prevent a decrease in and/or increase the barrier function of the skin.

### SUMMARY OF THE INVENTION

Surprisingly and unexpectedly, the inventors have demonstrated that the oil derived from akenes of Silybum marianum (L.) Gaertn. induces a cutaneous synthesis of lipids, in particular an endogenous synthesis of ceramides which allows, in addition to the nourishing and/or moisturizing effect for the skin, to increase the epidermal barrier function or to prevent an increase in this epidermal barrier function but also to increase protection of the skin against water loss and/or external aggressions.

According to a first aspect, the invention relates to the cosmetic use of an oil derived from akenes of Silybum marianum (L.) Gaertn. to prevent a decrease in and/or increase the epidermal barrier function.

The invention also relates to an oil derived from akenes of Silybum marianum (L.) Gaertn. for its use to prevent a decrease in and/or increase the epidermal barrier function.

The invention also relates to the use of an oil derived from akenes of Silybum marianum (L.) Gaertn. for the manufacture of a cosmetic composition to prevent a decrease in and/or increase the epidermal barrier function.

According to a second aspect, the invention relates to the cosmetic use of a cosmetic composition comprising at least one oil derived from akenes of Silybum marianum (L.) Gaertn. with at least one cosmetically acceptable excipient, to prevent a decrease in and/or increase the epidermal barrier function.

The invention also relates to a cosmetic composition comprising at least one oil derived from akenes of Silybum marianum (L.) Gaertn. with at least one cosmetically acceptable excipient for its use to prevent a decrease in and/or increase the epidermal barrier function.

According to a third aspect, the invention relates to a cosmetic method for preventing a decrease in and/or increasing the epidermal barrier function, comprising the administration to a person in need thereof, of an effective amount of an oil derived from akenes of Silybum marianum (L.) Gaertn. or a cosmetic composition comprising at least one oil derived from akenes of Silybum marianum (L.) Gaertn. with at least one cosmetically acceptable excipient.

### Definitions

In the present invention, the plant Silybum marianum (L.) Gaertn. may be designated in an abbreviated manner by the term Silybum marianum.

“Organic solvent immiscible with oil derived from akenes of Silybum marianum”, means, within the meaning of the present invention, an organic solvent which is not capable of mixing, or only partially mixing, with the oil derived from akenes of Silybum marianum, so that the mixture of the organic solvent and the oil derived from akenes of Silybum marianum gives a heterogeneous mixture in which at least two distinct phases can be observed.

By “non-polar solvent”, it is to understand, within the meaning of the present invention, a solvent selected for example from heptane, hexane, limonene, halogenated hydrocarbons (for example C₁ to C₃ chlorinated hydrocarbons such as chloroform or dichloromethane), supercritical CO₂, a mixture of supercritical CO₂ and ethanol, and mixtures of these solvents. Mention may also be made of 100% biosourced solvents such as for example EcoXtract LIPOCOS (supplier Pennakem Europa).

“Refining” means, within the meaning of the present invention, the steps of deodorization and/or bleaching and/or desolventization of Silybum marianum oil. Indeed, crude oils contain a certain number of constituents responsible for the taste and unpleasant odors and their poor conservation, which it may therefore be desirable to remove.

“Deodorization” means, within the meaning of the present invention, a treatment aimed at eliminating the odor or the taste of a vegetable oil. Deodorization can be done by heating the oil at high temperature (for example between 150 and 300° C., in particular between 150 and 250° C. or else between 150 and 200° C.), under vacuum, with injection of steam water.

“Bleaching” means, within the meaning of the present invention, a treatment aimed at reducing the color of the vegetable oil. Color measurement can be done according to Gardner’s measurement. The Gardner color scale is a visual comparison scale for the color of clear and transparent liquids. Bleaching can be obtained by contacting the oil with a bleaching earth (which will absorb the pigments (for example carotene, chlorophyll, etc.) responsible for the color) and heating (for example between 60 and 100° C.) which can be done under vacuum. Advantageously, a subsequent filtration step will allow to separate the oil and the used bleaching earth.

“Desolventization” means, within the meaning of the present invention, a treatment allowing to eliminate the solvent present in an oil. The desolventization can be carried out by distillation by heating the mixture under vacuum and/or by steam distillation under vacuum.

“C₁ to C₃ alcohol” means, within the meaning of the present invention, an R-OH alcohol whose R chain is a saturated, linear or branched hydrocarbon chain, comprising 1 to 3 carbon atoms. It may be methanol, ethanol, n-propanol or isopropanol, in particular methanol, ethanol or isopropanol. Preferably it will be isopropanol.

“Ambient temperature” means, within the meaning of the present invention, a temperature comprised from 15 to 40° C., preferably from 20 to 30° C., in particular of approximately 25° C. “Approximately”, means in the present description that the value concerned may be lower or higher by 10%, in particular by 5%, in particular by 2%, more particularly by 1%, than the indicated value.

“Topical application”, means within the meaning of the present invention, an application to the skin (including the scalp), and the mucous membranes.

“Epidermal barrier” means, within the meaning of the present invention, the cellular structures of the epidermis, in particular the tissue barrier formed by the corneocytes and the intercellular lipid cement.

“Epidermal barrier function” means, within the meaning of the present invention, the protective function of the epidermis, in particular against external aggressions, and the regulation of the transepidermal loss of water and ions.

“Cosmetically acceptable” means, within the meaning of the present invention, what is useful in the preparation of a cosmetic composition, which is generally safe, non-toxic and neither biologically nor otherwise undesirable and which is acceptable for cosmetic use, in particular by topical application to the skin.

### DETAILED DESCRIPTION OF THE INVENTION

According to a first aspect, the invention relates to the cosmetic use of an oil derived from akenes of Silybum marianum (L.) Gaertn. to prevent a decrease in and/or increase the epidermal barrier function.

According to a particular embodiment, the invention relates to the cosmetic use of an oil derived from akenes of Silybum marianum (L.) Gaertn. to strengthen the protection of the skin against water loss and/or external aggressions.

According to a particular embodiment, the invention relates to the cosmetic use of at least one oil derived from akenes of Silybum marianum (L.) Gaertn. to nourish and/or moisturize the skin, including the scalp, and/or the mucous membranes.

According to a particular embodiment, the invention relates to the cosmetic use of at least one oil derived from akenes of Silybum marianum (L.) Gaertn. to improve skin repair, by reinforcing or restoring the barrier function.

According to a particular embodiment, the invention relates to the cosmetic use of at least one oil derived from akenes of Silybum marianum (L.) Gaertn. to prevent and/or reduce tingling, itching, tightness, redness, irritation of the skin.

Oil derived from akenes of Silybum marianum will be used more particularly topically, in particular by application to the skin.

In the context of the present invention, the oil derived from akenes of Silybum marianum is obtained from the fruit (akene), the akenes can be whole or in pieces.

In the context of the present invention, the oil derived from akenes of Silybum marianum can be obtained by pressing the akenes or by extracting the akenes with a non-polar solvent.

In one embodiment of the present invention, the oil derived from akenes of Silybum marianum can be obtained by pressing the akenes of Silybum marianum, in particular by cold pressing, that is to say without heating, at ambient temperature, followed by a filtration step.

In a particular embodiment of the invention, the oil derived from akenes of Silybum marianum is obtained by pressing the akenes of Silybum marianum, followed by a step of filtration then of refining.

In a particular embodiment of the invention, the oil derived from akenes of Silybum marianum is obtained by pressing the akenes of Silybum marianum, followed by a filtration step, followed by an extraction step with a polar to moderately polar extraction solvent to remove polar compounds from the oil, the polar to moderately polar extraction solvent comprising, in particular consisting of, a hydrotropic aqueous solution, subcritical water or an organic solvent immiscible with the oil derived from akenes of Silybum marianum optionally mixed with water, then optionally a desolventization step.

Advantageously, the polar to moderately polar extraction solvent comprises, in particular consists of, an organic solvent immiscible with the oil derived from akenes of Silybum marianum optionally mixed with water.

The organic solvent immiscible with the oil derived from akenes of Silybum marianum may in particular be a C₁ to C₃ alcohol.

The polar to moderately polar extraction solvent may in particular be a C₁ to C₃ alcohol, optionally mixed with water.

The organic solvent immiscible with the oil derived from akenes of Silybum marianum, in particular a C₁ to C₃ alcohol such as methanol, ethanol or isopropanol, may be used in a mixture with water, in particular in an organic solvent/water volume ratio comprised between 80/20 and 100/0, in particular between 85/15 and 95/5, in particular of approximately 90/10.

The polar to moderately polar extraction solvent may in particular be selected from methanol, a methanol/water mixture, ethanol, an ethanol/water mixture, isopropanol and an isopropanol/water mixture.

According to a preferred embodiment, the polar to moderately polar extraction solvent will be methanol, an ethanol/water mixture in a volume ratio of approximately 90/10 or an isopropanol/water mixture in a volume ratio of approximately 90/10, preferably an isopropanol/water mixture in a volume ratio of approximately 90/10.

The extraction step with a polar to moderately polar extraction solvent of the oil derived from akenes of Silybum marianum will be carried out in particular by mixing the oil derived from akenes of Silybum marianum with the polar to moderately polar extraction solvent for 1 to 12 h and in particular at a temperature comprised between 15 and 25° C., in particular around 20° C.

The amount of polar to moderately polar extraction solvent used to carry out this extraction will advantageously be from 0.5 to 3 g, in particular from 1 to 3 g per 1 g of oil derived from akenes of Silybum marianum.

An extraction phase and a lipid phase (residual oil devoid of its polar compounds, also called exhausted residual oil) will then be obtained at the end of this extraction. The lipid phase will advantageously be separated from the extraction phase and recovered. It can then be desolventized, in particular under vacuum, to eliminate the residual polar to moderately polar extraction solvent and obtain an oil derived from akenes of Silybum marianum which is devoid of its polar constituents (free fatty acids, phytosterols, tocopherols).

In a particular embodiment of the invention, the oil derived from akenes of Silybum marianum is obtained by pressing the akenes of Silybum marianum, then extraction with a polar to moderately polar extraction solvent to remove polar compounds from the oil as detailed above, then desolventization as previously described, followed by deodorization.

In a particular embodiment of the invention, the oil derived from akenes of Silybum marianum is obtained by pressing the akenes of Silybum marianum, then extraction with a polar to moderately polar extraction solvent to remove polar compounds from the oil as detailed above, then desolventization as previously described, followed by bleaching.

In a particular embodiment of the invention, the oil derived from akenes of Silybum marianum is obtained by pressing the akenes of Silybum marianum, then extraction with a polar to moderately polar extraction solvent to remove polar compounds from the oil as detailed above, then desolventization as previously described, followed by deodorization and/or bleaching.

According to a second aspect, the invention relates to the use of a cosmetic composition comprising at least one oil derived from akenes of Silybum marianum with at least one cosmetically acceptable excipient, to prevent a decrease in and/or increase the epidermal barrier function.

According to a particular embodiment, the invention relates to the use of a cosmetic composition comprising at least one oil derived from akenes of Silybum marianum with at least one cosmetically acceptable excipient, to reinforce the protection of the skin against water loss and/or external aggressions.

According to a particular embodiment, the invention relates to the use of a cosmetic composition comprising at least one oil derived from akenes of Silybum marianum with at least one cosmetically acceptable excipient, to nourish and/or moisturize the skin, including the scalp, and/or the mucous membranes.

According to a particular embodiment, the invention relates to the use of a cosmetic composition comprising at least one oil derived from akenes of Silybum marianum with at least one cosmetically acceptable excipient, to improve skin repair, by reinforcing or restoring the barrier function.

According to a particular embodiment, the invention relates to the use of a cosmetic composition comprising at least one oil derived from akenes of Silybum marianum with at least one cosmetically acceptable excipient, to prevent and/or reduce tingling, itching, tightness, redness, irritation of the skin.

Advantageously the oil derived from akenes of Silybum marianum comprised in the cosmetic composition is prepared as described previously.

In a particular embodiment, the cosmetic composition according to the invention comprises between 0.01 and 40% by weight relative to the total weight of the composition, in particular between 0.1 and 20% by weight, in particular between 0.1 and 10% by weight, more particularly between 0.1 and 5% by weight, even more particularly between 0.1 and 2% by weight, and even more particularly between 0.5 and 1% by weight of oil derived from akenes of Silybum marianum relative to the total weight of the composition.

Preferably, the oil derived from akenes of Silybum marianum is present in the cosmetic composition at a content of approximately 1% by weight relative to the total weight of the composition.

According to a particular embodiment, the cosmetic composition according to the invention does not comprise the following ingredients named according to the INCI nomenclature: Enteromorpha compressa extract and/or Ocimum sanctum leaves extract. Thus, according to a particular embodiment, the cosmetic composition according to the invention does not comprise green algae extract and/or holy basil extract.

According to a particular embodiment, the cosmetic composition according to the invention does not comprise Momordica grosvenori fruit extract and/or Pseudopterogorgia elisabethae extract.

According to a particular embodiment, the cosmetic composition according to the invention does not comprise Defensil® which has the INCI name: Octyldodecanol (and) Echium plantagineum seed Oil (and) Helianthus Annuus (Sunflower) Seed Oil Unsaponifiables (and) Cardiospermum Halicacabum Flower/Leaf/Vine Extract (and) Tocopherol. Thus, according to a particular embodiment, the cosmetic composition according to the invention does not comprise a mixture of octyldodecanol, Echium plantagineum seed oil (also called plantain leaf blueweed oil), the unsaponifiable fraction of sunflower oil, an extract (in particular of flowers/leaves/stems) of Cardiospermum halicacabum (called heart pea vine, Indian heart, or Poc-poc), and tocopherol. According to a particular embodiment, the cosmetic composition according to the invention does not comprise Echium plantagineum seed oil, the unsaponifiable fraction of sunflower oil, and/or an extract (in particular of flowers/leaves/stems) of Cardiospermum Halicacabum.

According to a particular embodiment, the cosmetic composition according to the invention does not comprise green algae extract, holy basil extract, Momordica grosvenori fruit extract, Pseudopterogorgia elisabethae extract, Echium plantagineum seed oil, unsaponifiable fraction of sunflower oil, and/or an extract (in particular of flowers/leaves/stems) of Cardiospermum Halicacabum.

According to another embodiment according to the invention, the cosmetic composition according to the invention comprises the oil derived from akenes of Silybum marianum as the only active ingredient useful for nourishing and/or moisturizing the skin, and more particularly useful for preventing a decrease in and/or increasing the epidermal barrier function.

The cosmetic compositions according to the invention are advantageously intended for topical application, in particular by application to the skin.

The cosmetic compositions according to the invention may thus be in the forms which are usually known for topical administration, that is to say in particular lotions, milks, emulsions, serums, balms, masks, creams, dispersions, gels, foams or sprays.

Advantageously, it will be a balm and/or a milk.

The invention thus relates to cosmetic compositions according to one of the embodiments of the present invention, characterized in that they are in a form suitable for topical application.

The cosmetic compositions according to the invention, in addition to the oil derived from akenes of Silybum marianum, and a physiologically acceptable medium, may also contain surfactants, complexing agents, preservatives, stabilizing agents, emulsifiers, thickeners, gelling agents, humectants, emollients, trace elements, essential oils, perfumes, dyes, mattifying agents, chemical or mineral filters, moisturizing agents, thermal waters, etc.

According to a third aspect, the invention relates to a cosmetic method for preventing a decrease in and/or increasing the epidermal barrier function, comprising the administration, in particular topically, for example by application to the skin, to a person in need thereof, of an effective amount of an oil derived from akenes of Silybum marianum or of a cosmetic composition comprising at least one oil derived from akenes of Silybum marianum with at least one cosmetically acceptable excipient.

According to a particular embodiment, the invention relates to a cosmetic method for reinforcing the protection of the skin against water loss and/or external aggressions, comprising the administration, in particular topically, for example by application to the skin, to a person in need thereof, of an effective amount of an oil derived from akenes of Silybum marianum or of a cosmetic composition comprising at least one oil derived from akenes of Silybum marianum with at least one cosmetically acceptable excipient.

According to a particular embodiment, the invention relates to a cosmetic method for nourishing and/or moisturizing the skin, including the scalp, and/or the mucous membranes, comprising the administration, in particular topically, for example by application to the skin, to a person in need thereof, of an effective amount of an oil derived from akenes of Silybum marianum or of a cosmetic composition comprising at least one oil derived from akenes of Silybum marianum with at least one cosmetically acceptable excipient.

According to a particular embodiment, the invention relates to a cosmetic method for improving the skin repair, by reinforcing or restoring the barrier function, comprising the administration, in particular topically, by application to the skin, to a person in need thereof, of an effective amount of an oil derived from akenes of Silybum marianum or of a cosmetic composition comprising at least one oil derived from akenes of Silybum marianum with at least one cosmetically acceptable excipient.

According to a particular embodiment, the invention relates to a cosmetic method for preventing and/or reducing tingling, itching, tightness, redness, irritation of the skin, comprising the administration, in particular topically, by application on the skin, to a person in need threof, of an effective amount of an oil derived from akenes of Silybum marianum or of a cosmetic composition comprising at least one oil derived from akenes of Silybum marianum with at least one cosmetically acceptable excipient.

Advantageously the oil derived from akenes of Silybum marianum is prepared as described above and the cosmetic composition is as described above.

The following examples illustrate the invention without limiting the scope thereof.

### EXAMPLES

### Example 1: Preparation of the Oil Derived From Akenes of Silybum Marianum Obtained by Cold Pressing

Cold pressing of akenes of Silybum marianum then filtration on a filter press to obtain a first press raw oil derived from akenes of Silybum marianum (L.) Gaertn.

### Example 2: Preparation of the Refined Oil Derived From Akenes of Silybum Marianum Devoid of its Polar Compounds

This method is done in 8 steps:
Cold pressing of akenes of Silybum marianum then filtration on a filter press to obtain a first press raw oil derived from akenes of Silybum marianum (L.) Gaertn.
Extraction of the first press raw oil derived from akenes of Silybum marianum with an isopropanol/water mixture (90/10 v/v) with 1 weight of the isopropanol/water mixture for 1 weight of oil for 10 hours at 20° C.
Recovery of the lipid phase (exhausted residual oil)
Desolventization of the exhausted residual oil: elimination of the solvent by vacuum evaporation (distillation) then steam stripping under the following conditions:
   Step 1 - distillation:
   Distillation temperature: 90° C.
   Distillation duration: 2 hours
   Vacuum: 2 mbar progressive
   Stage 2 - stripping:
   Steam: around 7 kg/h
   Stripping temperature: 90° C.
   Stripping duration: 1 hour
   Vacuum: 2-20 mbar
Bleaching of exhausted and desolventized residual oil under the following conditions:
   Bleaching natural earth: Tonsil® 210 FF
   Bleaching temperature: 80° C.
   Bleaching duration: 45 minutes
   Vacuum: 10-20 mbar
Filtration on filter press
Deodorization under the following conditions:
   Deodorization temperature: 180° C.
   Deodorization duration: 2 hours
   Stripping: steam (about 7 kg/h)
   Vacuum: 2-20 mbar
Filtration on cartridge filter.

### Example 3: Effects of Oils From Akenes of Silybum Marianum on the Synthesis of Total Lipids and Ceramides on a Reconstructed Epidermis Model

The main function of the epidermis is to protect the body by forming a vital protective barrier against external aggressions and against the risk of dehydration. The stratum corneum, the outermost layer of the skin, is largely responsible for the barrier function. This stratum corneum is made up of corneocytes embedded in a lipid matrix, the very specific organization of which depends on the lipid composition. The latter is composed of free fatty acids, cholesterol and ceramides. Lipids form multiple layers superimposed on each other. In vitro experiments have demonstrated that the specific lipid composition of the stratum corneum alone allows this particular arrangement of lipids in lamellar bilayers (De Jager and al., J. Lipid res. 2005, 46, 2649-2656). These lipids play a key role in the barrier function of the skin.

Ceramides constitute a lipid family of great biological importance because they allow the cohesion of the stratum corneum and, consequently, the formation of the skin barrier. Biochemically, they are sphingolipids resulting from the amidation of sphingosine with a fatty acid. They can be free or covalently bound to proteins in the stratum corneum. Currently, 14 classes of ceramides have been identified and are named according to their chemical structure: ceramides can have a sphingosine (S), dihydrosphingosine (dS), phytosphingosine (P), or 6-hydroxysphingosine (H) basis to which is bound a ω-hydroxy (EO or O), α-hydroxy (A) or non-hydroxy (N) fatty acid with an alkyl chain of variable length. EO ceramides have a unique structure because they have a very long ω-hydroxy-acid chain of more than 34 carbon atoms bonded to a linoleic acid and will have a preponderant role in the organization of the lamellar bilayers of the stratum corneum and consequently on the barrier function.

The quantification of ceramides informs on the integrity or not of the barrier function and provides an enhancement of the dermo-cosmetic products.

A wide variety of ceramides enter into the lipid composition of the stratum corneum. They alone represent about half of the intercorneocyte lipids. Ceramides will have a key role in the organization of lamellar bilayers and in particular ultra-long chain esterified ceramides such as EOS, EOP, EOH ceramides (Bouwstra and al., Biochim Biophys Acta 1996, 1300(3), 177-186). The importance of esterified ceramides, due to their very long carbon chains, has been demonstrated on the lamellar repeat distance and in the arrangement of the chains (Kessner and al., Chem Phys Lipids, 2010, 163(1), 42-50). In addition, the polar heads carried by the ceramides, in particular CER EOS and CER EOP, exert a considerable influence on these structural properties required for a functional lipid matrix.

Non-esterified ceramides are in the majority and are also important for the barrier function but also for the hydration and nutrition of the skin. Studies have shown that, during winter, states of skin dryness have been correlated with a decrease in total ceramide levels and more particularly with NP and NH ceramide levels (Ishikawa and al., J. Cosmet Dermatol 2013, 12(1), 3-11). In patients with atopic dermatitis, there has also been reported a significant decrease in total ceramide levels and more particularly with NP, NS and NH ceramide levels and an inverse correlation with the measurement of transepidermal water loss indicating an alteration of the barrier function (Ishikawa and al., J Invest Dermatol, 2010, 130(10), 2511-2514).

The decrease in ultra-long chain esterified ceramides as well as the shortening of the chain lengths of free fatty acids and ceramides in general leads to a modification of the lipid organization towards a less dense state (Kessner and al., Skin Pharmacol Physiol 2008, 21(2), 58-74). This creates gaps in the lipid arrangement between corneocytes leading to a reduction in skin barrier function and increased skin permeability. Thus, an increase in these specific lipids induces an improvement in the skin barrier function.

The aim of this study is to assess the impact of oil derived from akenes of Silybum marianum on the synthesis of cutaneous lipids, and in particular on the synthesis of the major constituent ceramides of the stratum corneum from a lipid point of view and to evaluate the nutritive effect for the treatment and the improvement of the barrier function. The ceramides, the free fatty acids and the cholesterol of the stratum corneum are analyzed by High-Performance Thin-Layer Chromatography (or “HPTLC”). This rapid technique is widely used to separate complex mixtures such as lipids (Fuchs and al., J. Chromatography A 2011, 1218(19), 2754-2774), the method used has been the subject of a scientific publication (Jamin and al., Eur J Mass Spectrum, 2019, 25(3), 278-290). The oil effect is evaluated on 3 batches of reconstructed epidermis with n=3 (triplicate) per experimental condition and per batch. After treatment with the oil derived from akenes of Silybum marianum, the lipids of the stratum corneum are quantified by HPTLC in order to evaluate the impact of the treatment on the synthesis of cutaneous lipids.

### Method

The model used in this study is a model of reconstructed epidermis resulting from skin resections from cosmetic surgery according to the method described by Frankart and al. (Frankart and al., Exp. Dermatol. 2012, 21(11), 871-875).

The cells (keratinocytes) are isolated from skin resections, then placed in culture before being seeded on culture inserts immersed in culture medium then the culture inserts are placed at the air/liquid interface in an incubator at 37° C. in a humidified atmosphere with 5% CO₂, to form the stratum corneum

It takes 14 days to reform a reconstructed epidermis with an area of 0.6 cm². The culture medium is changed every 24 hours.

Three reconstructed epidermis are used per condition (control, oil derived from akenes of Silybum marianum obtained by cold pressing, and positive control).

Control: Tween® 20 at 0.01% in phosphate buffer pH 7.4 (PBS);
Oil derived from akenes of Silybum marianum:
   1^{st} series of experiments: oil obtained according to example 1, tested at 1% in Tween® 20 at 0.01% in phosphate buffer pH 7.4 (PBS);
   2^{nd} series of experiments: oil obtained according to example 2, tested at 1% in Tween® 20 at 0.01% in phosphate buffer pH 7.4 (PBS);
Positive control: Dexeryl® cream.

On the 9^{th} day of the protocol, the compounds to be tested are applied for the first time to the reconstructed epidermis (2 mg for the Dexeryl® cream, 5 µl of oil derived from akenes of Silybum marianum obtained by cold pressing at 1% in Tween® 20/PBS or 5 µl of Tween® 20/PBS for control by epithelium). An incubation of 24 hours is performed.

A second application (same conditions) is carried out on the 10^{th} day, with an incubation of 48 hours.

A third application (same conditions) is carried out on the 13^{th} day, with an incubation of 24 hours.

Finally, a fourth application (same conditions) is carried out on the 14^{th} day (completely reconstructed epithelium), with an incubation of 24 hours.

On the 15^{th} day, the reconstructed epidermis is removed from the culture inserts, the stratum corneum is isolated using trypsin from the rest of the epidermis. The stratum corneum is then extracted using organic solvents (mixture of chloroform and methanol) in order to collect the lipids that constitute it. These lipids are then concentrated under liquid nitrogen before HPLC analysis.

Ceramides, free fatty acids and cholesterol of the stratum corneum are analyzed by HPTLC. The analytical conditions are detailed below, in particular in Table 1.
Plate: Lichrospher® HPTLC Silica gel 60 F254S
Deposition: 6 mm wide, dried under nitrogen flow
Development: gradient: see Table 1
Post-derivatization: aqueous solution of copper sulphate (10% CuSO₄, 8% H₃PO₄, 5% MeOH) Detection: λ scanner: 450 nm.

TABLE 1

| Steps | Chloroform | Acetone | MeOH /Water/ Acetic acid (97/3/1v/v/v) | Distance (mm) |
|---|---|---|---|---|
| 1 | 81.5 | 4 | 14.5 | 20 |
| 2 | 81.7 | 4 | 14.3 | 30 |
| 3 | 82 | 4 | 14 | 42 |
| 4 | 83 | 4 | 13 | 46 |
| 5 | 84.5 | 4 | 11.5 | 54 |
| 6 | 85 | 4 | 11 | 57 |
| 7 | 86 | 4 | 10 | 59 |
| 8 | 87 | 4 | 9 | 67 |
| 9 | 88 | 4 | 8 | 75 |
| 10 | 90 | 5 | 5 | 83 |
| 11 | 100 | 0 | 0 | 90 |

### Results

Dexeryl® cream is known to increase lipid synthesis. Therefore, Dexeryl® cream was chosen as a positive control and was applied to the reconstructed epithelia at 2 mg/epithelium. Several classes of lipids are analyzed by HPTLC, free fatty acids, cholesterol derivatives (cholesterol oleate and cholesterol sulphate) and ceramides.

### 1^{st} Series of Experiments

In this first series of experiments, the positive control (Dexeryl® cream) induces, as expected, lipid synthesis, associated with an increase in free fatty acids, cholesterol derivatives and ceramides. These results validate the experimental conditions.

Oil derived from akenes of Silybum marianum according to example 1 has no effect on the synthesis of free fatty acids, induces a slight drop in total cholesterol (-4.3%) but this reduction does not reach the significance threshold. On the other hand, the oil derived from akenes of Silybum marianum according to example 1 significantly increases the synthesis of total ceramides (+36.1%). This result leads to the conclusion that this oil derived from akenes of Silybum marianum demonstrates an important nourishing effect for the skin.

Ceramides are present as the dominant lipids in the stratum corneum, and play a crucial role in barrier function and therefore limiting dehydration and water retention. Based on the important properties of ceramides, emphasis has been placed on different subclasses of ceramides, produced by the application of silybum marianum oil. Indeed, these ceramides were not present in the formulation, the ceramides found in the stratum corneum therefore correspond only to the ceramides produced by the skin.

Table 2 below shows the percentage of induction of the ceramides produced after the application of the oil derived from akenes of Silybum marianum according to example 1 compared to the control.

TABLE 2

| | | | | | | | | |
|---|---|---|---|---|---|---|---|---|
| CER AH | CER AP | CER NH | CER AS and EOH | CER AdS and OH | CER PN | CER EOP | CER NdS and NS | CER EOS |
| 15.3% | 24.4% | 25.8% | 39.0% | 45.3% | 44.3% | 63.7% | 46.4% | 32.0% |
| P<0.01 | P<0.05 | P<0.05 | P<0.05 | P<0.01 | P<0.05 | P<0.01 | P<0.001 | P<0.05 |

Thus, the oil derived from akenes of Silybum marianum, compared to the untreated reconstructed epidermis (control), induces a statistically significant synthesis of all the ceramides in this model of reconstructed epidermis. It is interesting to note that the increase in the synthesis of the ceramide CER EOS exceeds 30%, this ceramide being in reduced amount in the case of eczema and atopic dermatitis, and playing an important role in the barrier function and in particular the organization of lamellar bilayers. Moreover, the ceramides involved in the lamellar organization of lipids in the stratum corneum, that is to say the esterified ceramides with ultra-long chains, such as the EOS, EOH, and EOP ceramides, are significantly increased to more than 30% minimum. Non-esterified ceramides are also increased, such as NP and NS ceramides, which are the majority ceramides at the stratum corneum. Ceramide NP is the majority ceramide and contributes 8-13% to total ceramides (Van Smeden and al., J Lipid Res 2011, 52(6), 1211-1221). This ceramide plays an important role in the formation of lamellar bilayers with ultra-long chain esterified ceramides, as well as the AdS ceramide, which is also induced by the topical application of oil derived from akenes of Silybum marianum (Bouwstra and al., Biochim Biophys Acta 1996, 1300(3), 177-186). The ceramide NP with the ceramide NH are also implicated in skin dryness when the amounts of these ceramides decrease (Ishikawa and al., J Cosmet Dermatol 2013, 12(1), 3-11). The increase in the level of total ceramides and in particular ceramides which are major players in the barrier function such as the EOS, NP and NS ceramides, by applying oil derived from akenes of Silybum marianum, is very favorable for a reinforcing effect on the epidermal barrier function.

### 2^{nd} Series of Experiments

In this second series of experiments, the positive control (Dexeryl® cream) induces, as expected, a synthesis of lipids, in particular cholesterol derivatives and total ceramides. It should be noted that the synthesis of lipids is less than that found during the first series of experiments. These results still validate the experimental conditions.

Under these conditions, the oil derived from akenes of Silybum marianum according to example 2 increases the synthesis of total ceramides by about 13.6% relative to untreated reconstructed epidermis. These results therefore demonstrate a nourishing effect of this oil.

As in the 1^{st} series of experiments, the study focused on different classes of ceramides necessarily induced by the application of the products through the skin, since the ceramides are absent from the formulations. The results obtained by the application of the oil derived from akenes of Silybum marianum according to example 2 are summarized in Table 3.

Table 3 below shows the percentage of induction of ceramides produced by the application of oil derived from akenes of Silybum marianum according to example 2.

TABLE 3

| | | | | | | |
|---|---|---|---|---|---|---|
| CER AH | CER NH | CER AdS and OH | CER NP | CER EOP | CER NdS and NS | CER EOS |
| 5.9 | 25.8 | 30.5 | 12.7 | 12.8 | 20.7 | 25.7 |
| NSS | P<0.05 | P<0.01 | P<0.05 | P<0.05 | P<0.05 | P<0.05 |

Oil derived from akenes of Silybum marianum according to example 2 induces a statistically significant production of almost all the ceramides, in particular the ceramides which are major players in the barrier function.

All these results show that the oil derived from akenes of Silybum marianum has a nourishing effect but is also able to induce an endogenous synthesis of ceramides and therefore to improve the epidermal barrier function.

1-16. (canceled)

17. A cosmetic method for preventing a decrease in and/or increasing the epidermal barrier function, comprising the administration to a person in need thereof, of an effective amount of an oil derived from akenes of Silybum marianum (L.) Gaertn.

18. The method according to claim 17, for reinforcing the protection of the skin against water loss and/or external aggressions.

19. The method according to claim 17, for nourishing and/or moisturizing the skin and/or the mucous membranes.

20. The method according to claim 17, for improving the skin repair, by reinforcing or restoring the barrier function.

21. The method according to claim 17, for preventing and/or reducing tingling, itching, tightness, redness or irritation of the skin.

22. The method according to claim 17, wherein the oil derived from akenes of Silybum marianum (L.) Gaertn. is obtained by cold pressing akenes of Silybum marianum (L.) Gaertn. to obtain a cold pressed oil, followed by filtering the cold pressed oil.

23. The method according to claim 22, wherein a filtered oil is obtained by filtering the cold pressed oil and the filtering step is followed by extracting the filtered oil with a polar to moderately polar extraction solvent to give an extraction phase and a lipid phase, separating the lipid phase from the extraction phase, and recovering the lipid phase.

24. The method according to claim 23, wherein the polar to moderately polar extraction solvent is a C1 to C3 alcohol.

25. The method according to claim 23, wherein the recovering step is followed by desolventizing, and then optionally deodorizing and/or bleaching the lipid phase.

26. A cosmetic method for preventing a decrease in and/or increasing the epidermal barrier function, comprising the administration to a person in need thereof, of an effective amount of a cosmetic composition comprising an oil derived from akenes of Silybum marianum (L.) Gaertn. with at least one cosmetically acceptable excipient.

27. The method according to claim 26, for reinforcing the protection of the skin against water loss and/or external aggressions.

28. The method according to claim 26, for nourishing and/or moisturizing the skin and/or the mucous membranes.

29. The method according to claim 26, for improving the skin repair by reinforcing or restoring the barrier function.

30. The method according to claim 26, for preventing and/or reducing stinging, itching, tightness, redness or irritation of the skin.

31. The method according to claim 26, wherein the cosmetic composition contains from 0.1 to 2% by weight of the oil derived from akenes of Silybum marianum (L.) Gaertn. relative to the total weight of the cosmetic composition.
